(19) United States
(12) Patent Application Publication
(10) Pub. No.: US 2025/0297705 A1
(43) Pub. Date: Sep. 25, 2025

(54) DEVICE FOR DISPENSING ACTIVE INGREDIENTS OR CONVEYABLE SUBSTANCES IN A TARGETED, CONTROLLABLE, AND REGULATABLE MANNER USING A GAS GENERATING CELL

(71) Applicant: SIMATEC AG, Wangen an der Aare (CH)

(72) Inventors: Matthias MEIER, Thunstetten (CH); Hans GFELLER, Aarwangen (CH)

(21) Appl. No.: 18/863,814

(22) PCT Filed: Apr. 24, 2023
(86) PCT No.: PCT/EP2023/060666

§ 371 Date: Nov. 7, 2024

(30) Foreign Application Priority Data
May 23, 2022 (EP) 22174862.7
Jul. 11, 2022 (EP) 22184010.1
Jul. 11, 2022 (EP) 22184015.0

(51) Int. Cl. F16N 7/30; A61M 31/00; F16N 11/10

(52) U.S. Cl. CPC F16N 7/30; A61M 31/00; F16N 11/10; F16N 2270/20

(57) ABSTRACT

A conveyable medium, which may be gaseous, liquid, viscous, pasty, or pulverulent, is dispensed into an organism (human, animal, plant) or onto a technical system in a targeted, controlled, closed-loop controlled, and monitored manner by a displacement pumping principle using a gas produced by a gas generating cell. Conveyable media may be medicaments or active ingredients in a liquid, dissolved, or viscous form, as well as any type of supporting substance and liquid, and lubricants or protective agents (grease, oil) or reagents. The system includes the following parts: a) an operating, control, regulating, monitoring, and communication unit; b) a gas drive unit; c) an active ingredient reservoir; and d) a connection system.

The present invention relates to an apparatus for dispensing active ingredients or conveyable substances in a targeted, controllable, and regulatable manner by means of a conveying pressure built up by one or several gas generating cells.

Many apparatuses are known on the market for conveying small quantities of liquid or viscous substances according to a predetermined constant or variable dispensing quantity per time, which must be adhered to exactly, for dispensing active ingredient or conveyable substances into a human, animal or biological organism, predominantly in order to improve, change or maintain the state of health thereof and/or to influence bodily processes.

Apparatuses available on the market for dispensing active ingredients or other conveyable substances in human or veterinary medicine function electromagnetically, by means of spring pretensioning or on the basis of elastomers. Peristaltic pumps are also known. The electromechanical systems are characterized by precisely controllable dosing, but these systems are relatively large in comparison to the volume being conveyed. Both the spring-driven systems and the elastomer pumps are, on account of their design and the lack of electronics, imprecise and insufficiently reliable.

The gas generating cell technology underlying the present invention has not yet been used for dosing systems in the field of medicine in order to build a market-ready product that closes the gap between the precise but relatively large and expensive electromechanical systems and the simple but less precise and less expensive spring or elastomer pumps. However, it has not yet been possible to achieve the required reliability and precision with the possibilities hitherto available in gas generating cell technology.

The object underlying the present invention is therefore to specify an apparatus for dispensing active ingredients or conveyable substances in a targeted, controllable, and regulatable manner by means of a conveying pressure built up by a gas generating cell, such an apparatus being characterized by a compact size, its comparatively easily controllable conveying technology, and the conveying precision and controllable discharge that can be achieved thereby.

The technological progress over recent decades in the electronics industry and the current state of knowledge based on long-standing experience with the gas generating cell have contributed in very general terms to the achievement of this object, as a result of which it has now become possible to combine the advantages of the small size of the gas generating cell drive with corresponding intelligent electronics in order to build a very small, reliable, controllable, regulatable, monitorable and safe dosing system which achieves the necessary, significantly improved dosing precision in comparison to spring and elastomer pumps.

The advantages in comparison to the current systems lie in the smaller size, simpler design, increased mobility and independence, reduced complexity, improved precision, electronic regulatability, functional monitoring, digital communication and easier replenishment of the system. In comparison to gravity-driven infusion systems, the system is characterized by a position-independent mode of operation, which does not depend on the prevailing gravitational force and functions even in conditions of weightlessness.

The object underlying the invention is to dispense a conveyable medium, which can be gaseous, liquid, viscous or pulverulent, for example into an organism (human, animal, plant) or onto a technical system such as a machine or installation in a targeted, controlled, regulated, and monitored manner by means of a displacement pumping principle using a gas produced by one or more gas generating cells. Conceivable conveyable media are medicaments or active ingredients in liquid, dissolved or viscous form, but also any type of supporting substances and liquids which can be conveyed by the system, as well as lubricants (grease, oil) or reagents for use in the 17 technical field. The system should be inexpensive to produce and be suitable for economical mass production, and expendable and wear parts should be easy to replace. The operation and correct setup of the device as well as starting, pausing, or stopping the dispensing process should be easy, understandable, and designed such that the probability of faults is minimized and the safety of the patient is maximized, or a reliable and safe operation of the technical system is ensured.

This object is achieved by the system described below, as disclosed in the independent claim 1 and the subsequent claims 2 to 4.

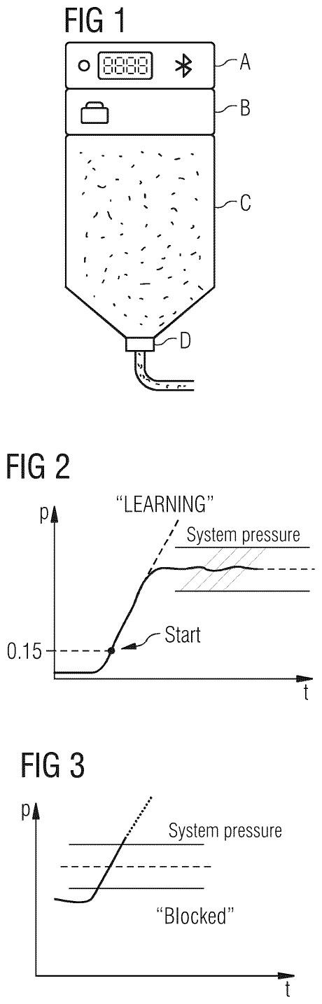

As shown in FIG. 1, the system consists substantially of the following parts:
a) an operating, control, regulating, monitoring and communication unit (A);
b) a gas drive unit (B);
c) a reservoir for active ingredient or conveyable substance (C); and
d) a connection system (D).
   A) Operating unit (operating, control, regulating, monitoring and communication unit)

The operating unit consists of a housing, a display (e.g. LCD), operating elements (e.g. buttons), an energy source (e.g. button battery or rechargeable battery), components for wireless communication, and electronic components which fulfill the functionalities described below.

The most important functions include one or several of the following properties:
a) a start function for an optimum ramp-up
b) an indirect regulation of the dispensing quantity by means of temperature, pressure and current measurement over time, and
c) a bolus function for reacting to higher dosages required for a short time.

The latter can be realized by controlled discharge current or by a defined gas accumulator with a trip valve. The time required until the next pressure build-up can be used to prevent, in a natural manner, an excessively frequent bolus dispensing.

The device can be easily set (dispensing rate ml/h, total quantity ml, etc.) and started via the operating elements and with the aid of the display.

The operating unit has electronics which enable wireless communication, for example with a PC, tablet or smartphone.

This makes it possible to set all necessary values precisely and also to program a temporal progression as required. In addition, this makes it possible to start and stop or pause the device contactlessly. Safe monitoring of the dispensing and networking of the patient, for example via blockchain technology, is also possible.

The system can also be extended by an apparatus for eliminating faults or problems, which can for example release a blockage by means of a pressure pulse. Such a pressure pulse can be realized with the aid of correspondingly connected valves, pistons, and springs or detectors.

A constant active ingredient dosing can be achieved by an intelligent regulation of the gas generating cell by means of pressure and temperature measurement with a corresponding regulation algorithm and a corresponding actuation of the gas generating cell.

The regulation of the dispensing volume/active ingredient dosing takes place indirectly via the pressure and temperature measurement. The general gas equation applies here: f(p, T, V)=constant.

The gas production, or discharge current in mAh is “normalized” during integration (normal pressure @ 20° C.). The gas production of the gas generating cells is known and controllable. As an alternative to this regulation method via pressure/temperature, a combination with a separate fill level measurement system is also conceivable, such as for example the measurement of the piston position or the measurement of the flow rate of the dispensing quantity.

Here, the gas diffusion through the housing/bag of the active ingredient to be conveyed is considered computationally via the diffusion function (ml) Vd=f(p, T, V, t). As a result, it is possible to calculate back to the current gas quantity or to the active ingredient (medicament) volume at any time via the pressure and temperature measurement.

After activation of the dispensing function (app, button, etc.), the regulating electronics and firmware can assume that there is a new, fully replenished active ingredient reservoir in the device. Here, all counters/integrators can be set to “zero,” in other words a fill level of 100% is assumed.

The size of the active ingredient reservoir can be identified automatically (HW coding on the reservoir) or is determined via the Uo voltage of the respective gas generating cell packet. The state of charge of the cell can be controlled simultaneously by way of voltage monitoring upon startup.

| Type | Uo(new) | Un(@180 ohms) | tn(180 ohms) |
|---|---|---|---|
| 100 ml | 0.5 . . . 1.5 V | <0.45 V | 15 min |
| 200 ml | 1.0 . . . 3.0 V | <0.90 V | 15 min |
| 500 ml | 2.5 . . . 5.0 V | <2.00 V | 15 min |

### Rapid Startup and Learning Phase

FIG. 2 outlines the initial startup of the active ingredient dosing device, during which the gas generating cells are discharged rapidly until a “single cell voltage” of below 0.4 V is established (conditioning). The gas generating cell does not begin the process of hydrogen gas production until this voltage point is reached. The rapid discharging of the gas generating cells now continues until a pressure of 0.15 bar is reached, at which point for example the piston friction of a conveying piston is overcome. The pressure increase (ascending gradient) can be monitored in the startup position, at which point a dead volume can also be compensated.

The pressure is increased until the pressure equilibrium (“dispense active ingredient”) is established. This pressure is recorded as the “system pressure” and is used as the control basis in subsequent evaluations. If the device assumes one or several fault states at a later time, the learning cycle could be initiated again. Furthermore, it is also possible to monitor whether the maximum system pressure (final pressure) is already exceeded in the startup phase and the device is therefore prevented from functioning faultlessly from the outset.

### “Blocked” Malfunction

If, despite continued gas production, no pressure equilibrium is established (as shown in FIG. 3) or the maximum, theoretical pressure is exceeded, the regulation can be trained so as to assume that the system is blocked, and consequently sends a fault indication or signals this fault on the device itself.

### “Leakage” Malfunction

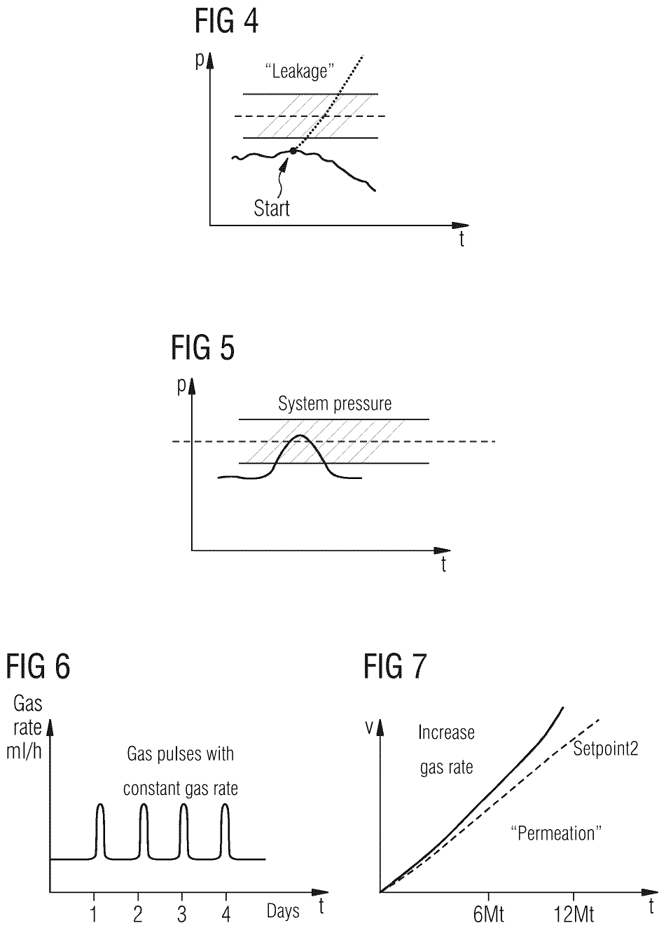

If, despite gas production, the pressure does not rise or even falls toward zero again, as shown in FIG. 4, the control can be embodied so that it assumes the presence of a leakage. As already described above, the active ingredient dosing device signals this fault and/or issues a fault indication.

### Pulsed Mode

FIG. 5 shows the case in which the gas generating cell is actuated in “pulsed mode” (approx. 1 ml in 22 min). Discharging at 180 ohms produces a gas rate of 2.7 ml/h. The pressure measurement signal should rise and then fall again once the active ingredient is flowing. The piston movement can be identified in this way. The stick-slip effect of the piston can be identified via the pressure measurement as a further indication of the piston movement or dosing function.

Pulsed Mode with Constant Minimal Gas Rate

It is also possible for the gas generating cell to be operated at a “constant minimal” gas rate and for the “pulses” to be overlaid, as shown in FIG. 6. This results in a more constant gas production, as much shorter “ramp-up phases” of the gas generating cell are required.

### Compensation of Gas Diffusion

The gas diffusion through a syringe housing or an exterior bag can also be compensated constantly by the control by an increasing gas rate being realized.

### B) Gas Drive Unit

The gas drive unit forms the actual drive of the dispensing system and can be connected to the operating unit (A). The gas drive unit consists at least of a housing, one or several gas generating cells, and contact elements. The gas drive unit is mechanically and electrically connected to the operating unit and the active ingredient reservoir (C). To increase the safety of a patient and to protect against manipulation, it is also conceivable for the gas drive unit to contain the entirety of the controlling, regulating and monitoring electronics, including sensors, communication elements (e.g. radio) and energy source (e.g. battery).

The gas generation is controlled via a programmable current discharge. The sensor system and the intelligent electronics, disposed either in the gas drive unit or the operating unit, enable a reliable dispensing of the active ingredient. It is also possible to pause the dispensing if necessary, for example if the patient feels unwell as a result of a pain relief dose which is set too high. Furthermore, the dispensing rate can be adjusted at any time during the dispensing process. For example, the dispensing quantity could also follow a pre-programmed dosing profile.

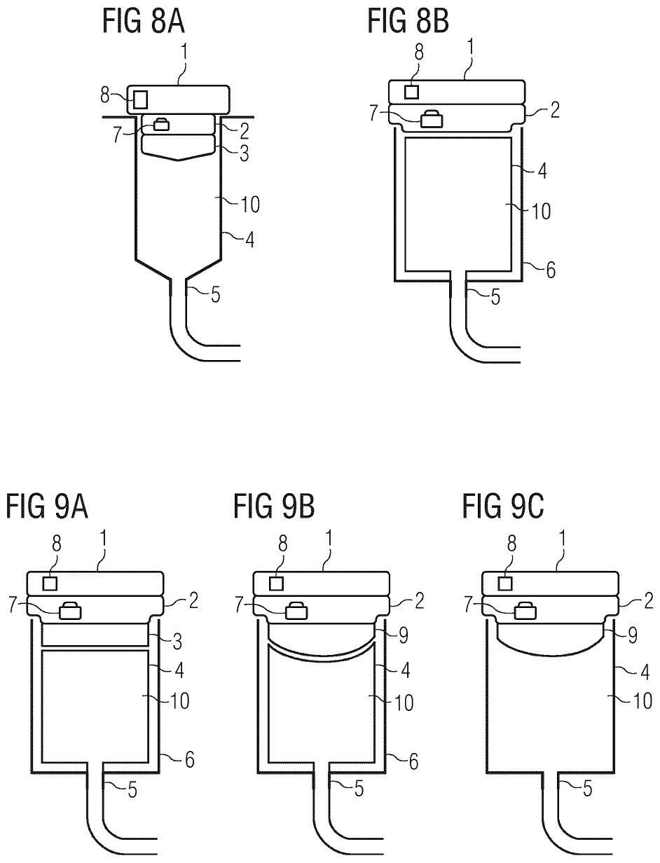

As shown in FIGS. 8 and 9, the gas drive unit could already be connected or welded to a housing 6 or an active ingredient container 4 at the production stage. However, it could also be connected thereto at the provision stage. The connection should be designed such that an undesired escape of gas is prevented and that the connection withstands the generated gas pressure required to discharge the active ingredient. A passive overpressure protection device which enables a controlled venting of the gas chamber in the event of critical overpressure could likewise be integrated in addition.

### C) Active Ingredient Reservoir

The active ingredient reservoir contains the medium to be conveyed. This is either pre-filled in advance or is replenished by the provider (pharmacist, healthcare service, etc.) or the patient themself. Filling is rendered as simple and safe as possible by a correspondingly configured structural design or a separate auxiliary apparatus, as a result of which it is easily possible to mix or blend medicaments shortly before dispensing them.

The active ingredient reservoir can be embodied in the form of a syringe available on the market (FIG. 8a). Here, the piston acts as the media separator between the gas and the medium to be conveyed, although further possible forms for separating the medium to be conveyed from the gas are also possible, such as for example “bag in bag”, “bag in fixed-shape housing”, or “bag in flexible-shape and fixed-volume housing” (FIG. 8b). An inflatable membrane or a type of bladder or balloon are however also technically conceivable as displacement mechanisms (FIG. 9b and FIG. 9c). The active ingredient reservoir should meet various application-specific requirements, in particular the material thereof should have optimum compatibility with the active ingredient and minimal or no gas diffusion. The 9 transition of gas through the media separator into the medium to be conveyed should however always be minimal and furthermore should not exceed any maximum values set. This can be achieved by a suitable material selection or coating or a multilayer design. A protection against light or UV radiation can also be implemented thereby.

The active ingredient reservoir can also be protected against heating up by an additional cold protection system or kept warm by a heating system.

It is also conceivable to be able to clip a bag into the active ingredient reservoir, which bag can then be pressed out via a piston, thus discharging the active ingredient (FIG. 9a).

### D) Connection System

A standard system which has already established itself on the market is preferably used as the connection system. This makes it easier for healthcare personnel, for example, to handle the device.

### LIST OF REFERENCE CHARACTERS

(1) Control unit
(2) Adapter
(3) Piston
(4) Active ingredient container
(5) Infusion instrument connection
(6) Housing
(7) Gas generating cell
(8) Electronics+battery if used+display if used+operating element if used
(9) Membrane/balloon/active ingredient

1-4. (canceled)

5. An apparatus for conveying a conveyable medium, the apparatus comprising:
a gas generating cell configured for generating gas for displacing the conveyable medium;
a containment configured for the conveyable medium in gaseous, liquid, viscous, pasty, or pulverulent form; and
the apparatus being configured for dispensing the conveyable medium into an organism, being a human, an animal or a plant, or onto a technical system in a targeted, controlled, regulated and monitored manner.

6. The apparatus according to claim 5, wherein the technical system is a machine or a technical installation.

7. The apparatus according to claim 5, wherein the conveyable medium is any one of a plurality of media selected from the group consisting of medicaments or active ingredients in liquid, dissolved or viscous form, any type of supporting substance and liquid to be conveyed by the system, and lubricants or protective agents or reagents for use in the technical system.

8. The apparatus according to claim 5, wherein the protective agents are grease or oil.

9. The apparatus according to claim 5, comprising an operating, control, regulating, monitoring and communication unit, a gas drive unit, an active ingredient reservoir, and a connection system.

10. The apparatus according to claim 9, wherein the operating, control, regulating, monitoring and communication unit is configured for carrying out one or more of the following:
a) a start function for an optimum ramp-up of a conveying flow;
b) an indirect regulation of the dispensing quantity by means of temperature, pressure, and current measurement over time, and
c) a bolus function for reacting to higher dosages required for a short time.
